(19) United States
(12) Patent Application Publication
(10) Pub. No.: US 2012/0136379 A1
(43) Pub. Date: May 31, 2012

(54) SURGICAL KNIFE AND METHOD FOR MAKING INCISION

(76) Inventor: Souheil Haddad, Bloomington, IN (US)

(21) Appl. No.: 13/306,605

(22) Filed: Nov. 29, 2011

Related U.S. Application Data
(60) Provisional application No. 61/417,577, filed on Nov. 29, 2010.

(51) Int. Cl. A61B 17/32

(52) U.S. Cl. 606/167

(57) ABSTRACT

A surgical knife may include a handle and a blade embedded in the handle. The handle may have a tissue-displacing member surrounding the blade.

### RELATED APPLICATIONS

This application claims the benefit of U.S. Provisional Application No. 61417577 filed Nov. 29, 2010.

### BACKGROUND OF THE INVENTION

The present invention generally relates to instruments for performing medical procedures and more particularly to surgical knives for making incisions.

When making an incision in a tissue layer such as a peritoneum or dural layer, there is a risk that a knife or scalpel blade may cause damage to underlying organs or tissue. In order to mitigate such risk, a practitioner must exercise great care to control a depth of cut during the process of making an incision. The practitioner must apply only enough pressure on a knife to cause the knife to penetrate and cut the layer of interest. Thus the practitioner may be required to not only guide the knife laterally in a desired direction, but he or she must also provide an appropriate vertical force that counterbalances weight of the knife and cutting force needed to penetrate the layer of interest while simultaneously avoiding injury to underlying organs or other tissue.

As can be seen, there is a need for a method and an instrument for making incisions that may reduce the risk of causing injury to underlying organs or tissue when making an incision.

### SUMMARY OF THE INVENTION

In one aspect of the present invention, a surgical knife may comprise a handle; and a blade embedded in the handle, the handle having a tissue-displacing member surrounding the blade.

In another aspect of the present invention, a method of making an incision in a tissue layer may comprise the steps of: positioning a blade of a knife in an incision plane with a rounded tip of a tissue-displacing member of the knife projecting below the incision plane; pressing on the knife to cause the tissue-displacing member to displace underlying tissue away from the incision plane and the tissue layer that is to be cut; and passing the blade through the tissue layer that is to be cut while maintaining downward pressure on the knife to maintain the tissue-displacing member against the underlying tissue to displace the underlying tissue from the incision plane.

These and other features, aspects and advantages of the present invention will become better understood with reference to the following drawings, description and claims.

### BRIEF DESCRIPTION OF THE DRAWINGS

FIG. 1 is a perspective view of a surgical knife in use in accordance with an embodiment of the invention;

FIGS. 2, 3 and 4 are perspective views of the knife of FIG. 1;

FIG. 5 is an elevation view of the knife of FIG. 1;

FIG. 6 is a cross-sectional view of the knife of FIG. 1 taken along the lines 6-6 of FIG. 5; and

FIG. 7 is a partial cross-sectional view of the knife of FIG. 1 showing an operational feature of the knife.

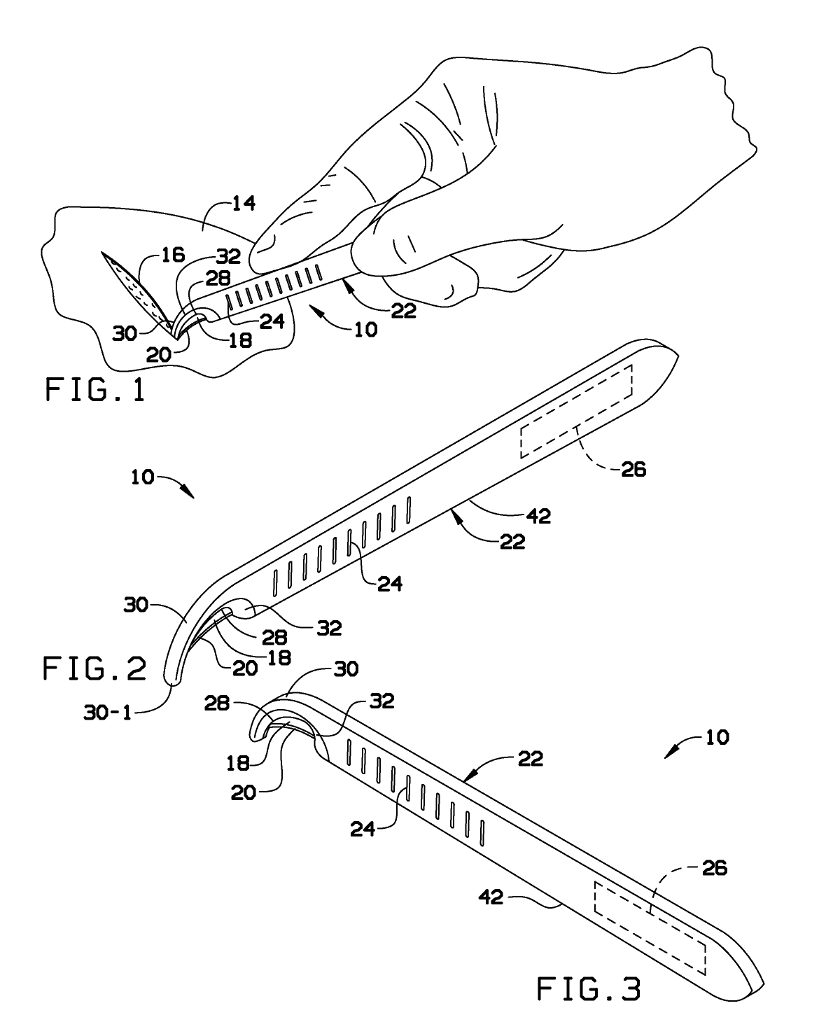

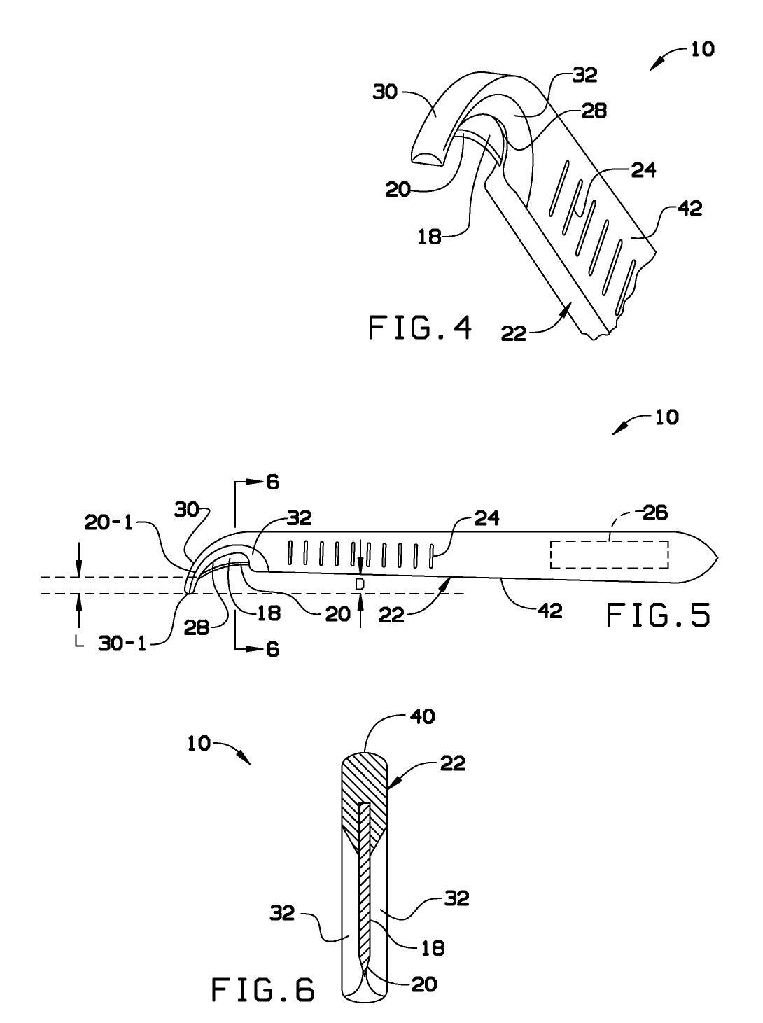

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Various inventive features are described below that can each be used independently of one another or in combination with other features.

Broadly, embodiments of the present invention generally provide a method and instrument for making surgical incisions through a tissue layer while holding underlying tissue away from an incision plane.

Referring to the Figures various views are shown of an exemplary embodiment of a surgical knife 10. The knife 10 may comprise a blade 18 with a blade cutting edge 20 and a handle 22 with handle grip ribs 24, a logo area 26, a blade recess 28, a tissue-displacing member 30 and a concave arc-shaped notch 32. The blade 18 may be a conventional scalpel blade that may have a curved or a straight cutting edge 20. The blade 18 may be positioned in the arc-shaped notch 32 of the handle 22. A portion of the cutting edge 20 may project below a lower surface 42 of the handle 22. The tissue-displacing member 30 may have a convex arc shape. An outer tip 30-1 of the member 30 may have a smooth rounded shape with a radius of between about 1.5 mm to 3 mm. The outer tip 30-1 may project below a forward end 20-1 of the blade cutting edge 20 by a distance L. Advantageously the distance L may be between about 1 mm to about 6 mm. The outer tip 30-1 may project a distance D below the bottom surface 42 of the handle 22. Advantageously, the distance D may be between about 7 mm to about 10 mm.

The knife 10 may be constructed with the blade 18 imbedded in the handle 22 as an injection molded assembly. In that regard, the knife 10 may be produced as a relatively inexpensive and disposable surgical instrument.

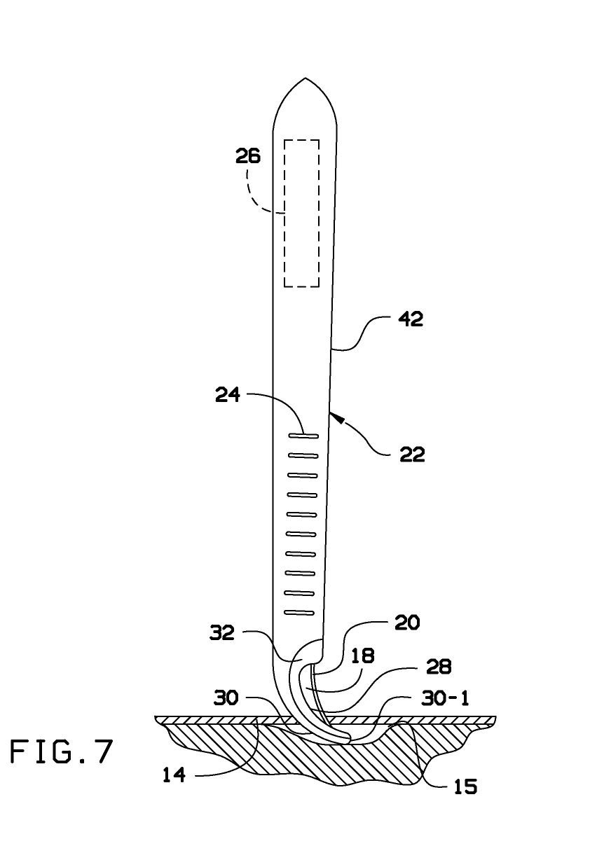

In operation, the knife 10 may be employed to safely make an incision in a tissue layer such as a peritoneum or dural layer without risk that the blade 18 may cause damage to underlying organs or tissue. Referring particularly to FIG. 7, it may be seen that tip 30-1 of the knife 10 may be inserted through an initial-entry opening in the tissue layer or membrane 14. The blade 20 may be then drawn across the membrane 14 to be cut. The tip 30-1 of the tissue-displacement member 30 may press against underlying tissue 15 to displace the tissue 15 from the incision plane. It may be seen that the knife 10 may safely make incisions while being either pulled or pushed along the incision plane.

It should be understood, of course, that the foregoing relates to exemplary embodiments of the invention and that modifications may be made without departing from the spirit and scope of the invention as set forth in the following claims.

What is claimed is:

1. A surgical knife comprising:
a handle; and
a blade embedded in the handle,
the handle having a tissue-displacing member surrounding the blade.

2. The knife of claim 1 wherein the tissue-displacing member has a convex arc shape.

3. The knife of claim 1 wherein the tissue-displacing member has a rounded outer tip that projects below a cutting edge of the blade.

4. The knife of claim 3 wherein the rounded tip has a radius between about 1.5 mm to about 3 mm.

5. The knife of claim 3 wherein the outer tip projects below a forward end of the cutting edge of the blade.

6. The knife of claim 3 wherein the outer tip projects below a forward end of the cutting edge of the blade by a distance L that is between about 1 mm to about 6 mm.

7. The knife of claim 3 wherein the outer tip projects below a bottom surface of the handle.

8. The knife of claim 1 wherein the blade is curved.

9. A method of making an incision in a tissue layer comprising the steps of:
positioning a blade of a knife in an incision plane with a rounded tip of a tissue-displacing member of the knife projecting below the incision plane;
pressing on the knife to cause the tissue-displacing member to displace underlying tissue away from the incision plane and the tissue layer that is to be cut; and
passing the blade through the tissue layer that is to be cut while maintaining downward pressure on the knife to maintain the tissue-displacing member against the underlying tissue to displace the underlying tissue from the incision plane.

10. The method of claim 9 further comprising the steps of:
making an initial-entry opening in the tissue layer; and
inserting the tissue-displacing member into the initial-entry opening.
